(19) United States
(12) Patent Application Publication
(10) Pub. No.: US 2012/0184630 A1
(43) Pub. Date: Jul. 19, 2012

(54) Odour Compounds

(76) Inventors: Philip Kraft, Duebendorf (CH); Urs Mueller, ThaMuang (TH)

(21) Appl. No.: 13/394,491

(22) PCT Filed: Sep. 10, 2010
(86) PCT No.: PCT/EP10/63288

§ 371 Date: Apr. 4, 2012

(30) Foreign Application Priority Data
Sep. 11, 2009 (GB) 0915993.0

(51) Int. Cl. A61K 8/37; C07C 69/24; C11D 3/50; A61Q 13/00; C07C 69/74; A61L 9/01

(52) U.S. Cl. 514/784; 560/124; 512/8; 510/102; 560/262; 512/26; 510/107; 560/123; 560/231; 512/22; 510/106

(57) ABSTRACT

A compound of formula (I)

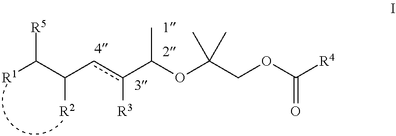

wherein R1 and R2 are independently selected from hydrogen, methyl, ethyl, propyl and isopropyl; or R1 and R2 together form a saturated or monounsaturated 5- or 6-membered hydrocarbon ring, as represented by the arcuate dotted line; R3 is selected from methyl and ethyl; R4 is selected from methyl, ethyl, propyl, isopropyl, cyclopropyl, butyl, isobutyl, cyclobutyl and cyclopentyl; R5 is selected from hydrogen and methyl, and the dotted line between positions 3″ and 4″ represents an optional double bond. The compounds have a musk odour and are useful in fine and functional fragrances.

This invention relates to compounds, to their use as odorants, and to fragrance compositions comprising them.

In the fragrance industry there is a constant demand for compounds having hedonic odour properties. Such compounds extend a perfumer's palette and result in greater product diversity for consumers.

In particular, there is demand for compounds that have musk odour characteristics. Such compounds are highly esteemed in perfumery and are perhaps some of the most versatile and common compounds found in fragrance compositions.

Historically, compounds having musk odour characteristics have been selected from one of three main structural classes: nitro arenes, polycyclic aromatics and macrocyclic compounds.

However, such compounds are often expensive, and in some places their use is restricted because of environmental concerns.

A new class of aliphatic carbonyl compounds having musk odour characteristics has been disclosed in PCT publication WO 2004/050595. However, whilst these compounds have addressed the problems detailed above, they are not as substantive as those compounds disclosed in the prior art. This is a significant drawback because substantivity is one of the most important characteristics of a compound with musk characteristics. One reason for this is their extensive use in laundry care products for which substantivity is a particularly important property.

Thus there is still a high demand for new substantive ecologically-benign compounds having musk odour characteristics.

Whilst it is known that there is a correlation between substantivity and the molecular weight of a compound, it was previously thought that any extensions to the structures of the compounds disclosed in WO 2004/050595 would lead to a dramatic loss of odour intensity (P. Kraft. W. Eichenberger. Eur. J. Org. Chem. 2004, 354). This was a logical assumption because it is well known that minor changes to the structure of a compound may result in the complete loss of musk odour (see for example, C. Sell (ed.), ‘The Chemistry of Fragrances—From Perfumer to Consumer’, 2^{nd} ed., RSC Publishing, Cambridge, 2006, p. 273).

It has now surprisingly been found that certain 2′-methyl-2″-(alkenyloxy)propyl and 2′-methyl-2′-(alkyloxy)propyl esters constitute new musk odorants that are ecologically benign and substansive.

The term “substantive” as used herein defines the duration of the perceptibility of a fragrance on a surface, for example, on skin, hair and fabric (G. Fráter, J. A. Bajgrowicz, P. Kraft. Tetrahedron 1998, 54, 7633). The substantivity of a compound is of profound economical and ecological importance. This is because the more odour that remains on a surface the more effective is the fragrance, and the less is washed out and the less burdened is the ecosystem.

A compound may be regarded as substantive if it has a medium to low vapour pressure and a low odour threshold (P. Müller, N. Neuner-Jehle, F. Etzweiler, Perfum. Flavor. 1993, 18, July/August, 49).

In general, a medium to low vapour pressure may be anything up to and including 40 μg/L, but more preferably it is anything up to and including 35 μg/L.

A low odour threshold may be anything up to and including 10 ng/L, but more preferably is anything up to and including 5 ng/L.

In a first aspect of the present invention there are provided compounds of formula (I)

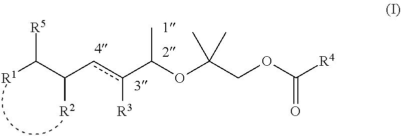

wherein R¹ and R² are independently selected from hydrogen, methyl, ethyl, propyl and isopropyl;
or R¹ and R² together form a saturated or monounsaturated 5- or 6-membered hydrocarbon ring, as represented by the arcuate dotted line;
R³ is selected from methyl and ethyl;
R⁴ is selected from methyl, ethyl, propyl, isopropyl, cyclopropyl, butyl, isobutyl, cyclobutyl and cyclopentyl;
R⁵ is selected from hydrogen and methyl, and the dotted line between positions 3″ and 4″ represents an optional double bond.

When present, the optional double bond between C-3″ and C-4″ may either be in (E)- or in (Z)-configuration.

More preferably,

R¹ is selected from hydrogen and methyl,
R² is selected from hydrogen and methyl,
or R¹ and R² together form a cyclohexenyl or cyclopentenyl ring;
R³ is selected from methyl and ethyl,
R⁴ is selected from ethyl, isopropyl, cyclopropyl and cyclobutyl,
R⁵ is selected from hydrogen and methyl, and
there is an (E)-configured double bond between C-3″ and C-4″.

Particularly preferred compounds of formula (I) are:
2′-Methyl-2′-(3″-methylhex-3″-en-2″-yloxy)propyl cyclopropanecarboxylate,
2′-(3″-ethyl-5″-methylhex-3″-en-2″-yloxy)-2′-methylpropyl cyclopropanecarboxylate, ethyl-5″-methylhex-3″-en-2″-yloxy)-2′-methylpropyl propionate, 2′-(3″-ethyl-5″-methylhex-3″-en-2″-yloxy)-2′-methylpropyl cyclobutanecarboxylate. 2′-(3″,6″-dimethylhept-3″-en-2″-yloxy)-2′-methylpropyl cyclopropanecarboxylate. 2′-(3″,5″-dimethylhept-3″-en-2″-yloxy)-2′-methylpropyl cyclopropanecarboxylate, and 2′-(4″-(cyclohex-3′″-enyl)-3″-methylbut-3″-en-2″-yloxy)-2′-methylpropyl isobutyrate.

A compound according to Formula I comprises at least one chiral centre, and as such may exist as a mixture of stereoisomers. Resolving stereoisomers adds to the complexity of manufacture and purification of these compounds, and so it is preferred to use the compounds as mixtures of their stereoisomers simply for economic reasons. However, if it is desired to prepare individual stereoisomers, this may be achieved according to methodology known in the art, e.g. preparative HPLC and GC or by stereoselective syntheses.

In the present invention, the use of the term “a compound” of formula (I)” may refer to both a racemic mixture and the individually isolated isomers.

The compounds of formula (I) may be prepared by known methods, described in open literature, using commercially-available reagents and solvents.

In particular, compounds of formula (I) may be prepared from α,β-unsaturated aldehydes, which may be prepared by standard aldol reactions from the corresponding aldehydes, in a straightforward 3-step process, consisting of:
(a) Grignard reaction of the respective α,β-unsaturated aldehyde with a methyl magnesium halide.
(b) Acid-catalyzed opening of isobutylene oxide, for example, aided by a Lewis acid such as tin tetrachloride.
(c) Esterification of the resulting alkyloxy alcohol by any standard esterification protocol, for example, the Steglich esterification or an industrially more viable method.

The resulting double bond between C-3″ and C-4″ can then optionally be hydrogenated catalytically, for example, in the presence of palladium on charcoal.

Alternatively, compounds of formula (I) may be prepared from the appropriate α,β-unsaturated methyl ketone. The process is the same as that described above, with the exception that, instead of a Grignard reaction in step (a), the required methyl carbinol can be prepared, for example, by hydride reduction. This is illustrated in Examples 5-7 of the present application.

A compound of formula (I) (within which term is included more than one such compound) may be used alone as a fragrance composition, or it may also be used in combination with one or more of the many known fragrance ingredients. There is therefore also provided a method of creating, enhancing or modifying a fragrance composition by using alone or in combination with at least one other fragrance ingredient a compound of formula (I). There is also provided a fragrance composition comprising a compound of formula (I).

Non-limiting examples of the extensive range of natural and synthetic fragrance molecules currently available include essential oils, alcohols, aldehydes and ketones, ethers and acetals, esters and lactones, macrocycles and heterocycles, and/or in admixture with one or more ingredients or excipients conventionally used in conjunction with odorants in fragrance compositions, for example carrier materials, and other auxiliary agents commonly used in the art.

Exemplary odorant molecules, which may be combined with the compounds of formula (I) include, but are not limited to:
ethereal oils and extracts, e.g. castoreum, costus root oil, oak moss absolute, geranium oil, jasmin absolute, patchouli oil, rose oil, sandalwood oil or ylang-ylang oil;
alcohols, e.g. citronellol, Ebanol™, eugenol, geraniol, Super Muguet™, linalool, phenylethyl alcohol, Sandalore™, terpineol or Timberol™.
aldehydes and ketones, e.g. α-amylcinnamaldehyd, Georgywood™, hydroxycitronellal, Iso E Super®, Isoraldeine®, Hedione®, maltol, methyl cedryl ketone, methylionone or vanillin;
ether and acetals. e.g. Ambrox™, Cassyrane, geranyl methyl ether, rose oxide or Spirambrene™.
esters and lactones, e.g. benzyl acetate, cedryl actetate, γ-decalactone, Helvetolide®, Sylkolide, γ-undecalactone or vetivenyl acetate.
macrocycles, e.g. ambrettolide, ethylene brassylate or Exaltolide®.
heterocycles, e.g. isobutylchinoline.

Examples of carrier materials and other auxiliary agents commonly used in conjunction with fragrance compositions can be found in, for example, in S. Arctander, ‘Perfume and Flavour Materials of Natural Origin’, Elizabeth, N.J., 1960, S. Arctander, ‘Perfume and Flavour Chemicals’, Vol. I and II, Allured Publishing Corporation, Carol Stream, 1994, and J. M. Nikitakis (Ed.), ‘CTFA Cosmetic Ingredient Handbook’ 1st ed., The Cosmetic. Toiletry and Fragrance Association, Inc., Washington. 1988.

The quantities in which compounds of formula (I) may be added to fragrance compositions may vary within wide limits and depend, inter alia, on the nature of the fragrance composition, on the effect desired, and on the nature and quantity of any other components of the fragrance composition. It may also depend on the purpose for adding compounds of formula (I) to a fragrance composition, for example, for perfuming a composition, for masking another odour, or for enhancing the fragrance. It is well within the purview of the person skilled in the art to decide on suitable quantities of compounds of formula (I) to incorporate into a fragrance composition depending on the end use and effect desired.

Typical, non-limiting, concentrations of said compounds in fragrance compositions are 0.005-25 weight percent, particularly 0.05-10 weight percent, more particularly 0.1-5 weight percent.

The compounds of formula (I) may be added into fragrance compositions in neat form, or in a solvent, or they may first be entrapped with an entrapment material for example, polymers, capsules, microcapsules, nanocapsules, liposomes, precursors, film formers, absorbents such as for example by using carbon or zeolites, or they may be chemically bound to substrates which are adapted to release the flavour molecule upon application of an exogenous stimulus such as light, enzyme, or the like, and then mixed with the fragrance composition.

The compounds of formula (I) can be added to all manner of fragrance compositions. However, due to their unique character, the compounds of formula (I) are especially well suited for use in fruity-floral fragrance compositions, in fragrance compositions with transparent “Cashmere wood” notes, or in fragrances compositions in which “white musk” accords are desired.

The use of compounds of formula (I) in fragrance compositions is illustrated in Example 8-11.

In a further aspect of the present invention, there is additionally provided a method of creating, modifying or enhancing a fragrance application, comprising the incorporation therein of a compound of formula (I) as a fragrance ingredient.

Compounds of formula (I) can be added to fragrance applications by using conventional techniques to directly admix a compound of formula (I) into the fragrance application, or alternatively to admix a fragrance composition comprising a compound of formula (I) into a fragrance application.

The quantities in which compounds of formula (I) may be added to fragrance applications may vary within wide limits and depend, inter alia, on the nature of the fragrance application, on the effect desired, the purpose for adding compounds of formula (I) to a fragrance composition, for example perfuming a composition, masking another odour, enhancing the fragrance, and on the nature and quantity of any other components of the fragrance application It is well within the purview of the person skilled in the art to decide on suitable quantities of compounds of formula (I) to incorporate into a fragrance applications depending on the end use and effect desired.

The compounds of the formula (I), may be used in a broad range of fragrance applications. e.g. in any field of fine and functional perfumery, such as perfumes, household products, laundry products, body care products and cosmetics.

The invention is now further described with reference to the following non-limiting examples.

### EXAMPLE 1

### (3″E)-2′-Methyl-2′-(3″-methylhex-3″-en-2″-yloxy)propyl cyclopropanecarboxylate

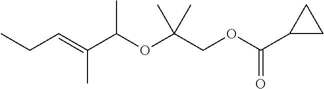

At room temp. under N₂ atmosphere, a 3 M solution of methyl magnesium chloride (160 mL, 480 mmol) in THF was added dropwise with stirring over a period of 45 min to a solution of commercial-grade (2E)-2-methyl pent-2-enal (47.1 g, 480 mmol) in THF (960 mL). Upon complete addition the temp. had reached 38° C., and the stirred reaction mixture was refluxed for 2 h. The heating source was then replaced by a cooling bath, and the reaction was quenched between 0-10° C. by slow addition of sat. aq. NH₄Cl (250 ml), followed by water (250 ml). The aqueous layer was removed and extracted with Et₂O (500 mL), the combined organic extracts were washed with brine (200 ml), dried (Na₂SO₄), and concentrated on a rotatory evaporator at 60° C./500 mbar. The resulting residue (92.0 g) was purified by silica-gel flash chromatography (FC, pentane/Et₂O, 4:1, R_{f}=0.25) to provide 28.8 g (53%) of (3E)-3-methylhex-3-en-2-ol as a colorless liquid.

At 0° C. under N₂, a solution of SnCl₄ (1.23 g, 4.71 mmol) in toluene (3.0 ml) was added to (3E)-3-methylhex-3-en-2-ol (35.0 g, 307 mmol), upon which the colour changes to pale yellow. Into this reaction mixture, a solution of isobutylene oxide (9.05 g, 126 mmol) in (3E)-3-methylhex-3-en-2-ol (6.20 g, 243 mmol) was added dropwise with stirring over a period of 90 min, at such a rate as to keep the temp. between 20-24° C. with occasional cooling. After complete addition, stirring was continued a further 30 min at room temp., prior to pouring the reaction mixture upon ice (100 g) and adding sat. aq. NH₄Cl (100 mL). The product was extracted with Et₂O (2×200 ml), and the combined ethereal extracts were washed with water (100 ml) and brine (100 ml). After drying (Na₂SO₄) and filtration of the ethereal solution over Celite®, the solvent was evaporated under reduced pressure to provide a mixture (43.7 g) of (3′E)-2-methyl-2-(3′-methylhex-3′-en-2′-yloxy)propan-1-ol and the starting (3E)-3-methylhex-3-en-2-ol. This starting material was removed by distillation at 55° C./50 mbar, the resulting residue (17.6 g) purified by silica-gel FC (pentane/Et₂O, 9:1) to provide 4.82 g (21%) of (3′E)-2-methyl-2-(3′-methylhex-3′-en-2′-yloxy)propan-1-ol as a colorless liquid.

At room temp., (3′E)-2-methyl-2-(3′-methylhex-3′-en-2″-yloxy)propan-1-ol (2.18 g, 11.7 mmol), cyclopropanecarboxylic acid (1.01 g, 11.7 mmol), and 4-(dimethylamino)pyridine (DMAP, 140 mg, 1.15 mmol) were dissolved with stirring in Et₂O (10 ml). To this stirred solution was added at 0° C. a solution of N,N′-dicyclohexylcarbodiimide (DCC, 2.66 g, 12.9 mmol) in Et₂O (5 ml), upon which the temp. rose to 4° C., and a colourless precipitate was formed. After 10 min the cooling bath was removed, and stirring was continued at ambient temp. overnight. The formed precipitate was vacuum filtrated and washed with Et₂O. The combined filtrates were concentrated under reduced pressure, and the resulting residue (3.76 g) purified by silica-gel FC (pentane/Et₂O, 19:1, R_{f}=0.31) to furnish 1.66 g (56%) of the odoriferous title compound (3′E)-2-methyl-2-(3′-methylhex-3′-en-2′-yloxy)propyl cyclopropanecarboxylate.

IR (ATR): ν=1162/1066 (s, νC—O), 1730 cm⁻¹ (s, νO—C═P).—¹H NMR (CDCl₃): δ=0.83-0.88 (m, 2H, 3-H_{b}), 0.93 (t, J=7.5 Hz, 3H, 6″-H₃), 0.99-1.02 (m, 2H, 3-Hₐ), 1.14 (d, J=6.5 Hz, 3H, 1″-H₃), 1.18 (s, 6H, 2′-Me₂), 1.59 (br. s, 3H, 3″-Me), 1.64 (m_{c}, 1H, 2H), 1.99 (m_{c}, 2H, 5″-H₂), 3.91 (d, J=11.0 Hz, 1H, 1′-H_{b}), 3.97 (d, J=11.0 Hz, 1H), 4.04 (q, J=6.5 Hz, 1H, 2″-H), 5.32 (t, J=7.0 Hz, 1H, 4″-H).—¹³C NMR (CDCl₃): δ=8.3 (2t. C-3). 11.2 (q. 3″-Me), 12.9 (d, C-2), 13.8 (q, C-6″), 20.7 (t, C-5″), 22.5 (q, C-1″). 23.6/23.7 (2q, 2′-Me₂). 69.8 (t, C-1′), 73.4 (d, C-2″), 74.4 (s, C-2′), 126.1 (d, C-4″), 138.2 (s, C-3″). 174.6 (s, C-1).—MS (70 eV): m/z=254 (1) [M⁺], 239 (1) [M⁺-CH₃], 141 (21) [C₈H₁₃O₂^{+]}, 114 (5) [C₇H₁₄O⁺], 97 (77) [C₇H₁₃⁺], 87 (2) [C₄H₇O₂⁺], 69 (100) [C₄H₅O⁺], 55 (55) [C₄H₇⁺], 41 (28) [C₃H₅⁺].

Odour description: musky, powdery, spicy with peppery aspects and some reminiscence to Cashmeran (1,1,2,3,3-pentamethyl-2,3,6,7-tetrahydro-1H-inden-4(5H)-one).

### EXAMPLE 2

### (3″E)-2′-(3″-Ethyl-5″-methylhex-3″-en-2″yloxy)-2′-methylpropyl cyclopropanecarboxylate

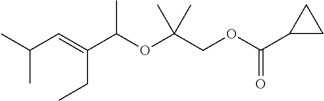

Following the protocol of Example 1, Grignard reaction of methyl magnesium chloride with 2-ethyl-3-isopropylacrolein (BASF, GB Patent 734000) provided (3E)-3-ethyl-5-methylhex-3-en-2-ol, which (40.0 g, 281 mmol) was reacted with isobutylene oxide (7.03 g, 97.5 mmol) in the presence of a solution of SnCl₄ (960 mg, 3.69 mmol) in toluene (2.0 ml) to afford 11.3 g of (54%) (3′E)-2-(3′-ethyl-5′-methylhex-3′-en-2′-yloxy)-2-methylpropan-1-ol after silica-gel FC (pentane/Et₂O, 9:1, R_{f}=0.11).

Also following the procedure detailed in Example 1, (3′E)-2-(3′-ethyl-5′-methylhex-3′-en-2′-yloxy)-2-methylpropan-1-ol (2.25 g, 10.5 mmol) was esterified with cyclopropanecarboxylic acid (4.10 g, 47.6 mmol) in the presence of 4-(dimethylamino)pyridine (DMAP, 600 mg, 4.92 mmol) and N,N′-dicyclohexylcarbodiimide (DCC, 10.8 g, 52.4 mmol) in Et₂O (60 ml) to furnish 1.60 g (54%) of the odoriferous title compound (3″E)-2′-(3″-ethyl-5″-methylhex-3″-en-2″yloxy)-2″-methylpropyl cyclopropanecarboxylate after work-up and purification by silica-gel FC (pentane/Et₂O, 19:1, R_{f}=0.37).

IR (ATR): ν=1162/1076 (s, νC—O), 1730 cm⁻¹ (s, νO—C═O).—¹H NMR (CDCl₃): δ=0.83-0.88 (m. 2H, 3-H_{b}), 0.94 (d, J=6.5 Hz. 6H, 5″-Me₂). 0.99-1.02 (m, 2H, 3-Hₐ). 1.04 (t, J=7.5 Hz, 3H, 2′″-H₃), 1.17 (d, J=6.5 Hz, 3H, 1″-H₃), 1.18 (s, 6H, 2′-Me₂), 1.64 (m_{c}, 1H. 2-H). 1.98-2.14 (m, 2H, 1′″-H₂), 2.51 (dsept, J=10.0, 6.5 Hz, 1H), 3.90 (d, J=11.0 Hz, 1H, 1′-H_{b}), 4.00 (d, J=11.0 Hz, 1H), 4.03 (q, J=6.5 Hz, 1H, 2″-H), 5.16 (d, J=10.0 Hz, 1 H, 4″-H).—¹³C NMR (CDCl₃): δ=8.3 (2t, C-3). 12.9 (d, C-2), 15.1 (q. C-2″), 20.3 (t, C-1′″), 22.9/23.2/23.3/23.4/23.8 (5q, 2′-, 5″-Me₂, C-1″), 26.6 (d, C-5″), 70.0 (t, C-1′), 72.3 (d, C-2″), 74.5 (s, C-2′), 132.4 (d, C-4″), 141.7 (s, C-3″), 174.7 (s, C-1).—MS (70 eV): m/z=282 (1) [M⁺], 267 (1) [M⁺-CH₃], 141 (35) [C₈H₁₃O₂⁺], 125 (13) [C₉H₁₇⁺], 109 (6) [C₈H₁₃⁺], 98 (6) [C₇H₁₄⁺], 83 (19) [C₆H₁₁⁺], 69 (100) [C₄H₅O⁺], 55 (17) [C₄H₇⁺], 41 (18) [C₃H₅⁺].

Odour description: musky, powdery with slightly waxy facets and some reminiscence to macrocyclic musks.

### EXAMPLE 3

### (3″E)-2′-(3″-Ethyl-5″-methylhex-3″-en-2″-yloxy)-2′-methylpropyl propionate

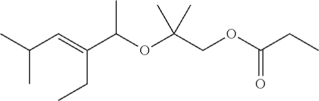

Following the procedure detailed in Example 2, (3′E)-2-(3′-ethyl-5′-methylhex-3′-en-2′-yloxy)-2-methylpropan-1-ol (2.25 g, 10.5 mmol) was esterified with propionic acid (2.70 g. 36.4 mmol) in the presence of 4-(dimethylamino)pyridine (DMAP, 440 mg, 3.61 mmol) and N,N′-dicyclohexylcarbodiimide (DCC, 7.50 g, 36.4 mmol) in Et₂O (60 ml) to furnish 1.54 g (54%) of the odoriferous title compound (3″E)-2′-(3″-ethyl-5″-methylhex-3″-en-2″-yloxy)-2′-methylpropyl propionate after work-up and purification by silica-gel FC (pentane/Et₂O, 19:1, R_{f}=0.30).

IR (ATR): ν=1167/1085 (s, νC—O), 1741 cm⁻¹ (s, νO—C═O).—¹H NMR (CDCl₃): δ=0.93 (d, J=6.5 Hz, 6H, 5″-Me₂), 1.03 (t, J=7.5 Hz, 3H, 2′″-H₃), 1.158 (t, J=7.5 Hz, 3H, 3-H₃), 1.163 (d, J=6.5 Hz, 3H, 1″-H₃), 1.17 (s, 6H, 2′-Me₂), 1.98-2.14 (m, 2H, 1′″-H₂), 2.36 (q, J=7.5 Hz, 2H, 2-H₂), 2.51 (dsept, J=10.0, 6.5 Hz, 1H 5″-H), 3.91 (d. 1=11.0 Hz, 1H, 1′-H_{b}) 4.00 (d, J=11.0 Hz, 1H), 4.03 (q, J=6.5 Hz, 1H, 2″-H). 5.16 (d, J=10.0 Hz, 1H, 4″-H).—¹³C NMR (CDCl₃): δ=9.1 (q, C-3), 15.1 (q, C-2′″), 20.2 (1, C-1′″), 22.9/23.2/23.3/23.4/23.8 (5q, 2′-, 5″-Me₂, C-1″), 26.5 (d, C-5″), 27.6 (t, C-2), 70.0 (t, C-1′), 72.3 (d, C-2″), 74.5 (s, C-2′), 132.4 (d, C-4″), 141.7 (s, C-3″), 174.2 (s, C-1).—MS (70 eV): m/z=255 (1) [M⁺-CH₃], 141 (13) [C₈H₁₃O₂⁺], 129 (35) [C₇H₁₃O₂⁺], 125 (17) [C₉H₁₇⁺], 109 (11) [C₈H₁₃⁺], 99 (9) [C₇H₅⁺], 83 (27) [C₆H₁₁⁺], 69 (54) [C₄H₅O⁺], 57 (100) [C₃H₅O⁺], 41 (14) [C₃H₅⁺]. 29 (9) [C₂H₅⁺].

Odour description: powerful musky, powdery with fruity-floral, slightly ambrette seed-like aspects and some reminiscence to macrocyclic musks.

### EXAMPLE 4

### (3″E)-2′-(3″-Ethyl-5″-methylhex-3″-en-2″-yloxy)-2′-methylpropyl cyclobutanecarboxylate

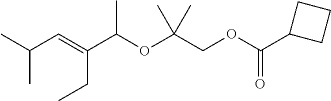

According to the procedure detailed in Example 3, (3′E)-2-(3′-ethyl-5′-methylhex-3′-en-2′-yloxy)-2-methylpropan-1-ol (2.25 g, 10.5 mmol) was esterified with cyclobutanecarboxylic acid (2.70 g, 36.4 mmol) in the presence of 4-(dimethylamino)pyridine (DMAP, 600 mg, 4.91 mmol) and N,N′-dicyclohexylcarbodiimide (DCC. 10.8 g, 52.4 mmol) in Et₂O (60 ml) to furnish 2.30 g (65%) of the odoriferous title compound (3″E)-2″-(3″-ethyl-5″-methylhex-3″-en-2″-yloxy)-2′-methylpropyl cyclobutanecarboxylate after work-up and purification by silica-gel FC (pentane/Et₂O, 19:1, R_{f}=0.41).

IR (ATR): ν=1163/1057 (s, νC—O), 1733 cm⁻¹ (s, νO—C═O).—¹H NMR (CDCl₃): δ=0.93 (d, J=6.5 Hz, 6H, 5″-Me₂), 1.03 (t, J=7.5 Hz, 3H, 2′″-H₃), 1.16 (d, J=6.5 Hz, 3H, 1″-H₃), 1.17 (s, 6H, 2′-Me₂), 1.89-1.94 (m, 1H, 4-H_{b}), 1.97-2.14 (m, 3H. 4-H_{b}, 1′″-H₂), 2.19-2.23 (m, 2H, 3-,5-H_{b}), 2.26-2.36 (m, 2H, 3-,5-Hₐ), 2.51 (dsept, J=10.0, 6.5 Hz, 1H, 5″-H), 3.17 (quintd, J=8.5, 1.0 Hz, 1H, 2″-H), 3.91 (d, J=11.0 Hz, 1H, 1′-H_{b}), 4.00 (d, J=11.0 Hz, 1H, 1′-Hₐ), 4.04 (q, J=6.0 Hz, 1H, 2″-H), 5.15 (d, J=10.0 Hz, 1H, 1∝-H).—¹³C NMR (CDCl₃): δ=15.1 (q, C-2′″), 18.4 (t, C-4), 20.2 (t, C-1′″), 22.9/23.2/23.3/23.4/23.8 (5q, 2′-, 5″-Me₂. C-1″), 25.2 (2t, C-3,-5), 26.5 (d, C-5″), 38.2 (d, C-2). 69.9 (t, C-1′). 72.3 (d, C-2″). 74.5 (s, C-2′), 132.4 (d, C-4″), 141.7 (s, C-3″), 175.2 (s, C-1).—MS (70 eV): m/z=281 (1) [M⁺], 155 (35) [C₉H₁₅O₂⁺], 141 (26) [C₉H₁₇O⁺], 125 (27) [C₉H₁₇⁺], 109 (12) [C₈H₁₃⁺], 101 (2) [C₅H₉O₂⁺], 95 (11) [C₇H₁₁⁺], 83 (100) [C₆H₁₁⁺], 69 (67) [C₄H₅O⁺], 55 (100) [C₃H₅O⁺], 41 (15) [C₃H₅⁺].

Odour description: musky, powdery, with slightly green, sappy and somewhat waxy facets.

### EXAMPLE 5

### (3″E)-2′-(3″,6″-Dimethylhept-3″-en-2″-yloxy)-2′-methylpropyl cyclopropanecarboxylate

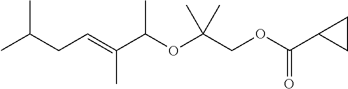

Over a period of 1 h, a solution of commercial-grade (3E)-3,6-dimethylhept-3-en-2-one (23.5 g. 168 mmol) in Et₂O (85 ml) was added dropwise at room temp. under N₂ atmosphere to a stirred suspension of lithium aluminium hydride (1.75 g, 46.1 mmol) in Et₂O (170 ml). Upon complete additional the temp. had risen to 35° C., and the reaction mixture was then heated to reflux for a further 2 h, prior to quenching by careful addition of water (30 ml) between 0-5° C. When the evolution of H₂ gas had ceased, 5 N aq. HCl (30 ml) was added, and the product was extracted with Et₂O (2×100 ml). The combined ethereal extracts were washed with water (100 ml) and brine (50 ml), dried (Na₂SO₄) and concentrated to dryness on the rotary evaporator. The resulting residue (23.7 g) was purified by silica-gel FC (pentane/Et₂O, 4:1. R_{f}=0.32) to provide 22.1 g (93%) of (3E)-3,6-dimethylhept-3-en-2-ol as a colorless liquid.

Following the protocol of Example 1, (3E)-3,6-dimethylhept-3-en-2-ol (21.0 g, 148 mmol) was reacted with isobutylene oxide (3.70 g, 51.3 mmol) in the presence of a solution of SnCl₄ (500 mg, 1.92 mmol) in toluene (1.3 ml) to afford 7.51 g (68%) of (3′E)-2-(3′,6′-dimethylhept-3′-en-2′-yloxy)-2-methylpropan-1-ol.

Also following the procedure detailed in Example 1, (3′E)-2-(3′,6′-dimethylhept-3′-en-2′-yloxy)-2-methylpropan-1-ol (3.74 g, 17.4 mmol) was esterified with cyclopropanecarboxylic acid (6.14 g, 29.7 mmol) in the presence of 4-(dimethylamino)pyridine (DMAP, 870 mg, 7.12 mmol) and N,N′-dicyclohexylcarbodiimide (DCC. 16.2 g, 78.5 mmol) in Et₂O (105 ml) to provide 3.33 g (71%) of the odoriferous title compound (3″E)-2′-(3″,6″-dimethylhept-3″-en-2″-yloxy)-2′-methylpropyl cyclopropanecarboxylate after work-up and purification by silica-gel FC (pentane/Et₂O, 19:1, R_{f}=0.25).

IR (ATR): ν=1162/1072 (s, νC—O). 1730 cm⁻¹ (s, νO—C═O).—¹H NMR (CDCl₃): δ=0.83-0.85 (m, 2H, 3-H_{b}), 0.87 (d, J=7.0 Hz, 3H, 6″-Me_{b}), 0.88 (d, J=6.5 Hz, 3H, 6″-Meₐ), 0.98-1.02 (m, 2H, 3-Hₐ), 1.15 (d, J=6.5 Hz, 3H, 1″-H₃). 1.180 (s, 3H, 1″-Me_{b}), 1.183 (s, 3H, 2′-Meₐ), 1.54-1.69 (m, 2H, 2-, 6″-H), 1.59 (s, 3H, 3″-Me), 1.83-1.91 (m, 2H, 5″-H₂). 3.91 (d, J=11.0 Hz, 1H, 1′-H_{b}). 3.99 (d, J=11.0 Hz, 1H, 1′-Hₐ), 4.06 (q, J=6.5 Hz, 1H, 2″-H), 5.36 (t, J=7.5 Hz, 1H, 4″-H).—¹³C NMR (CDCl₃): δ=8.3 (2t, C-3). 11.5 (q. 3″-Me). 12.9 (d, C-2), 22.3/22.4/22.7/23.6/23.8 (5q. 2′-, 6″-Me₂, C-1″), 28.8 (d, C-6″). 36.6 (t, C-5″). 68.8 (t, C-1′), 73.5 (d, C-2″), 74.4 (s, C-2′), 123.3 (d, C-4″), 139.5 (s, C-3″) 174.6 (s, C-1).—MS (70 eV): m/z=267 (1) [M⁺-CH₃], 196 (1) [M₊-C₄H₆O₂], 141 (29) [C₈H₁₃O₂⁺], 125 (12) [C₉H₁₇], 109 (5) [C₈H₁₃⁺], 83 (10) [C₆H₁₁⁺], 69 (100) [C₄H₅O⁺]. 55 (12) [C₄H₇⁺], 41 (20) [C₃H₅⁺].

Odour description: dry, musky, powdery with slightly green aspects.

### EXAMPLE 6

### (3″E)-2′-(3″,5″-1-Dimethylhept-3″-en-2″-yloxy)-2′-methylpropyl cyclopropanecarboxylate

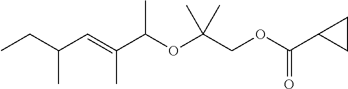

Following the hydride reduction of Example 5, (3E)-3,5-dimethylhept-3-en-2-one (20.0 g, 143 mmol) was reduced with lithium aluminium hydride (1.49 g. 39.3 mmol) in Et₂O (210 ml) to provide 18.9 g (93%) of (3E)-3,5-dimethylhept-3-en-2-ol as a colourless liquid.

Following the epoxide opening reaction of Example 1, (3E)-3,5-dimethylhept-3-en-2-ol (18.9 g, 133 mmol) was reacted with isobutylene oxide (3.32 g, 46.0 mmol) in the presence of a solution of SnCl₄ (450 mg. 1.74 mmol) in toluene (1 ml) to furnish 4.96 g (50%) of (3′E)-2-(3′,5′-dimethylhept-3′-en-2′-yloxy)-2-methylpropan-1-ol after silica-gel FC (pentane/Et₂O, 4:1, R_{f}=0.31).

Following the Steglich esterification of Example 1, (3′E)-2-(3%5′-dimethylhept-3′-en-2″-yloxy)-2-methylpropan-1-ol (1.23 g, 5.75 mmol) was esterified with cyclopropanecarboxylic acid (2.01 g, 23.3 mmol) in the presence of 4-(dimethylamino)pyridine (DMAP, 290 mg. 2.37 mmol) and N,N′-dicyclohexylcarbodiimide (DCC, 5.31 g, 25.7 mmol) in Et₂O (35 ml) to provide 1.40 g (86%) of the odoriferous title compound (3″E)-2′-(3″,5′-dimethylhept-3″-en-2″-yloxy)-2′-methylpropyl cyclopropanecarboxylate after work-up and purification by silica-gel FC (pentane/Et₂O, 98:2, R_{f}=0.09).

IR (ATR): ν=1162/1072 (s, νC—O), 1730 cm⁻¹ (s, νO—C═O).—¹H NMR (CDCl₃): δ=0.82/0.86 (d, J=9.0 Hz, 3H, 6″-Me), 0.83-0.85 (m, 2H, 3-H_{b}), 0.89/0.91 (d, J=7.0 Hz, 3H. 5″-Me), 0.99-1.03 (m, 2H, 3-Hₐ), 1.15/1.16 (d, J=6.5 Hz, 3H, 1″-H₃), 1.182 (s, 3H, 2′-Me_{b}), 1.184 (s, 3H, 2′-Meₐ). 1.21 (m_{c}, 1H, 6″-H_{b}). 1.32 (m_{c}. 1H, 6″-Hₐ). 1.60 (s, 3H, 3″-Me), 1.63 (m_{c}. 1H, 2-H), 2.23 (mₑ, 1H, 5″-H), 3.89/3.90 (d, 0.1=11.0 Hz, 1H, 1′-H_{b}), 3.99/4.01 (d, J=11.0 Hz, 1H, 1′-H_{b}), 4.04/4.05 (q, J=6.5 Hz, 1H, 2″-H), 5.08 (t, J=10.0 Hz, 1H, 4″-H).—¹³C NMR (CDCl₃): δ=8.3 (t, C-3), 11.5/11.6 (q, 3″-Me), 11.9/12.0 (q, C-7″), 12.9 (d, C-2), 20.3/20.6 (q, 5″-Me), 22.70/22.73/23.46/23.58/23.74/23.81 (3q, 2′-Me₂, C-1″). 30.2/30.3 (t, C-6″). 33.6 (d, C-5″), 69.8/69.9 (t, C-1′), 73.5/73.6 (s, C-2″), 74.4/74.5 (d, C-2″), 130.7/130.8 (d, C-4″), 137.6/137.7 (s, C-3″), 174.6 (s, C-1).—MS (70 eV): m/z=267 (1) [M⁺-CH₃], 141 (31) [C₈H₁₃O₂⁺], 125 (11) [C₉H₁₇⁺], 109 (6) [C₈H₁₃⁺], 83 (16) [C₆H₁₁⁺], 69 (100) [C₄H₅O⁺], 55 (14) [C₄H₇⁺], 41 (19) [C₃H₅⁺].

Odour description: powerful musky, powdery, with slightly aromatic, fresh, spicy elements and green nuances.

### EXAMPLE 7

### (3″E)-2′-(4″-(Cyclohex-3′″-enyl)-3″-methylbut-3″-en-2″-yloxy)-2′-methylpropyl isobutyrate

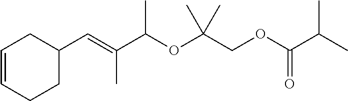

Following the hydride reduction of Example 5, (E)-4-(cyclohex-3-enyl)-3-methylbut-3-en-2-one (35.0 g, 213 mmol) was reduced with lithium aluminium hydride (2.22 g, 58.6 mmol) in Et₂O (320 ml) to provide 34.5 g (97%) of (E)-4-(cyclohex-3-enyl)-3-methylbut-3-en-2-ol as a colourless liquid after purification by silica-gel FC (pentane/Et₂O, 4:1, R_{f}=0.27).

Following the epoxide opening reaction of Example 1, (E)-4-(cyclohex-3-enyl)-3-methylbut-3-en-2-ol (34.5 g, 208 mmol) was reacted with isobutylene oxide (5.18 g, 71.8 mmol) in the presence of a solution of SnCl₄ (710 mg. 2.73 mmol) in toluene (1.7 ml) to furnish 8.96 g (52%) of (3′E)-2-(4′-(cyclohex-3″-enyl)-3′-methylbut-3′-en-2′-yloxy)-2-methylpropan-1-ol after silica-gel FC (pentane/Et₂O, 4:1, R_{f}=0.29).

Following the Steglich esterification of Example 1, (3′E)-2-((4′-(cyclohex-3″-enyl)-3′-methylbut-3′-en-2′-yl)oxy)-2-methylpropan-1-ol (1.80 g, 7.55 mmol) was esterified with isobutyric acid (3.07 g, 34.8 mmol) in the presence of 4-(dimethylamino)pyridine (DMAP, 420 mg, 3.44 mmol) and N,N′-dicyclohexylcarbodiimide (DCC, 7.89 g, 38.2 mmol) in Et₂O (57 ml) to provide 2.29 g (98%) of the odoriferous title compound (3″E)-2′-(4″-(cyclohex-3″-enyl)-3″-methylbut-3″-en-2″-yloxy)-2′-methylpropyl isobutyrate after work-up and purification by silica-gel FC (pentane/Et₂O, 19:1. R_{f}=0.06).

IR (ATR): ν=1736 cm⁻¹ (s, νO—C═O), 1162/1072 (s, νC—O).—¹H NMR (CDCl₃): δ=1.15 (d, J=6.5 Hz, 3H, 1″-H₃), 1.176 (br. s, 6H, 2′-Me₂), 1.184/1.186 (d, J=7 Hz, 6H, 2-Me₂), 1.35 (m_{c}, 1H, 6′″-H_{b}), 1.62/1.63 (s, 3H, 3″-Me), 1.65 (m_{c}, 1H, 2′″-H_{b}), 1.75 (m_{c}, 1H, 6′″-Hₐ),1.99-2.08 (m, 3H, 5′″-H₂), 2.45 (m_{c}, 1H, 1′″-H), 2.58 (sept. J=7.0 Hz, 1H, 2-H), 3.91 (d, J=11.0 Hz, 1H, 1′-H_{b}), 3.98/3.99 (d, J=11.0 Hz, 1H, 1′-Hₐ), 4.05 (q, J=6.5 Hz, 1H, 2″-H), 5.24 (d, J=9.0 Hz, 1H, 4″-H), 5.67 (m_{c}, 2H, 4′″-H).—¹³C NMR (CDCl₃): δ=11.40/11.41 (q, 3″-Me), 18.92/18.97 (2q, C-3), 22.6/23.53/23.54/23.7/23.8 (3q. 2′-Me₂, C-1″), 24.70/24.72 (t, C-5′″), 28.4/28.6 (t, C-6′″), 31.1/31.4 (t, C-2′″), 32.1/32.2 (d, C-1′″), 34.1 (d, C-2), 69.7 (t, C-1′), 73.4/73.5 (d, C-2″), 74.5 (s, C-2′), 126.2/126.3/126.8/126.9 (2d, C-3′″, -4′″), 129.5/129.6 (d, C-4″), 137.9 (s, C-3″), 176.8 (s, C-1).—MS (70 eV): m/z=293 (1) [M⁺-CH₃], 165 (22) [C₁₁H₁₇O⁺], 149 (40) [C₁₁H₁₇⁺],143 (44) [C₈H₁₅O₂⁺], 121 (8) [C₉H₁₃⁺], 107 (17) [C₈H₁₁⁺], 93 (24) [C₇H₉⁺], 79 (37) [C₆H₇⁺], 71 (100) [C₄H₇O⁺]. 43 (61) [C₃H₅⁺].

Odour description: musky, powdery, with fresh, green aspects and slightly waxy facets.

### EXAMPLE 8

### Sensual floriental feminine fine fragrance with (3″E)-2′-(3″-ethyl-5″-methylhex-3″-en-2″-yloxy)-2′-methylpropyl propionate

| Compound/Ingredient | parts by weight 1/800 |
|---|---|
| Ambrofix ((3aR,5aS,9aS,9bR)-dodecahydro-3a,6,6,9a- | 2 |
| tetramethylnaphtho[2,1-b]furan) | |
| Cepionate (cis-enriched methyl 2-(3′-oxo-2′-pentylcyclopentyl)acetate) | 55 |
| Citronellyl formiate | 3 |
| Dipropylene glycol (DPG) | 160 |
| Ebanol (3-methyl-5-(2′,2′,3′-trimethylcyclopent-3′-enyl)pent-4-en-2-ol) | 25 |
| Ethyl maltol (2-ethyl-3-hydroxy-4H-pyran-4-one) at 10% in DPG | 5 |
| Georgywood (cis-1-(1′,2′,3,4′,5′,6′,7′,8′-octahydro- | 120 |
| 1′,2′,8′,8′-tetramethyl-2-naphthalenyl)ethanone) | |
| Isoraldeine 95 (3-methyl-4-(2′,6′,6′-trimethyl-2′-cyclohexenyl)but-3-en-2-one) | 45 |
| Jasmine absolute Egypt pure | 2 |
| Linalool synthetic | 45 |
| Mandarin oil pure (yellow) | 30 |
| Pepperwood (3,7-dimethylocta-1,6-dien-3-yl dimethylcarbamate) | 10 |
| Pink pepper oil (Baies Roses) CO₂ extract pure | 3 |
| Sandalore (3-methyl-5-(2′,2′,3′-trimethylcyclopent-3′-enyl)pentan-2-ol) | 75 |
| Sandela (4-(5′,5′,6′-trimethylbicyclo[2.2.1]heptan-2′-yl)cyclohexanol) | 55 |
| Vanillin | 5 |
| (3″E)-2′-(3″-Ethyl-5″-methylhex-3″-en- | 160 |
| 2″-yloxy)-2′-methylpropyl propionate | |
| | 800 |

At 20%, (3″E)-2′-(3″-ethyl-5″-methylhex-3″-en-2″-yloxy)-2′-methylpropyl propionate provides to this feminine fragrance warmth and sensuality with its pleasant and characteristic powerful musk odour. Its powdery, ambrette, seed-like tonality reminiscent of macrocyclic musks blends very well with Isoraldeine 90, while its fruity-floral facets enhance the character of Cepionate.

### EXAMPLE 9

### Sensual floriental feminine fine fragrance with (3″E)-2′-(3″-ethyl-5″-methylhex-3″-en-2″-yloxy)-2′-methylpropyl cyclobutanecarboxylate

| Compound/Ingredient | parts by weight 1/800 |
|---|---|
| Ambrofix ((3aR,5aS,9aS,9bR)-dodecahydro-3a,6,6,9a- | 2 |
| tetramethylnaphtho[2,1-b]furan) | |
| Cepionate (cis-enriched methyl 2-(3′-oxo-2′-pentylcyclopentyl)acetate) | 55 |
| Citronellyl formiate | 3 |
| Dipropylene glycol (DPG) | 160 |
| Ebanol (3-methyl-5-(2′,2′,3′-trimethylcyclopent-3′-enyl)pent-4-en-2-ol) | 25 |
| Ethyl maltol (2-ethyl-3-hydroxy-4H-pyran-4-one) at 10% in DPG | 5 |
| Georgywood (cis-1-(1′,2′,3′,4′,5′,6′,7′,8′-octahydro- | 120 |
| 1′,2′,8′,8′-tetramethyl-2-naphthalenyl)ethanone) | |
| Isoraldeine 95 (3-methyl-4-(2′,6′,6′-trimethyl-2′-cyclohexenyl)but-3-en-2-one) | 45 |
| Jasmine absolute Egypt pure | 2 |
| Linalool synthetic | 45 |
| Mandarin oil pure (yellow) | 30 |
| Pepperwood (3,7-dimethylocta-1,6-dien-3-yldimethylcarbamate) | 10 |
| Pink pepper oil (Baies Roses) CO₂ extract pure | 3 |
| Sandalore (3-methyl-5-(2′,2′,3′-trimethylcyclopent-3′-enyl)pentan-2-ol) | 75 |
| Sandela (4-(5′,5′,6′-trimethylbicyclo[2.2.1]heptan-2′-yl)cyclohexanol) | 55 |
| Vanillin | 5 |
| (3″E)-2′-(3″-ethyl-5″-methylhex-3″-en-2″-yloxy)- | 160 |
| 2′-methylpropyl cyclobutanecarboxylate | |
| | 800 |

The use of 20% of (3″E)-2′-(3″-ethyl-5″-methylhex-3″-en-2″-yloxy)-2′-methylpropyl cyclobutanecarboxylate instead of the corresponding propionate enhances with its sweet musk character the vanillic side, while its green, waxy, and sappy characteristics blend very well with the jasmine absolute to increase the overall naturalness of this composition.

### EXAMPLE 10

### Combined use of (3″E)-2′-(3″-ethyl-5″-methylhex-3″-en-2″-yloxy)-2′-methylpropyl propionate and (3″E)-2′-(3″-ethyl-5″-methylhex-3″-en-2″-yloxy)-2′-methylpropyl cyclobutanecarboxylate

| Compound/Ingredient | parts by weight 1/800 |
|---|---|
| Ambrofix ((3aR,5aS,9aS,9bR)-dodecahydro-3a,6,6,9a- | 2 |
| tetramethylnaphtho[2,1-b]furan) | |
| Cepionate (cis-enriched methyl 2-(3′-oxo-2′-pentylcyclopentyl)acetate) | 55 |
| Citronellyl formiate | 3 |
| Dipropylene glycol (DPG) | 160 |
| Ebanol (3-methyl-5-(2′,2′,3′-trimethylcyclopent-3′-enyl)pent-4-en-2-ol) | 25 |
| Ethyl maltol (2-ethyl-3-hydroxy-4H-pyran-4-one) at 10% in DPG | 5 |
| Georgywood (cis-1-(1′,2′,3′,4′,5′,6′,7′,8′-octahydro- | 120 |
| 1′,2′,8′,8′-tetramethyl-2-naphthalenyl)ethanone) | |
| Isoraldeine 95 (3-methyl-4-(2′,6′,6′-trimethyl-2′-cyclohexenyl)but-3-en-2-one) | 45 |
| Jasmine absolute Egypt pure | 2 |
| Linalool synthetic | 45 |
| Mandarin oil pure (yellow) | 30 |
| Pepperwood (3,7-dimethylocta-1,6-dien-3-yl dimethylcarbamate) | 10 |
| Pink pepper oil (Baies Roses) CO₂ extract pure | 3 |
| Sandalore (3-methyl-5-(2′,2′,3′-trimethylcyclopent-3′-enyl)pentan-2-ol) | 75 |
| Sandela (4-(5′,5′,6′-trimethylbicyclo[2.2.1]heptan-2′-yl)cyclohexanol) | 55 |
| Vanillin | 5 |
| (3″E)-2′-(3″-Ethyl-5″-methylhex-3″-en- | 80 |
| 2″-yloxy)-2′-methylpropyl propionate | |
| (3″E)-2′-(3″-ethyl-5″-methylhex-3″-en-2″-yloxy)- | 80 |
| 2′-methylpropyl cyclobutanecarboxylate | |
| | 800 |

The use of 10% each of the (3″E)-2′-(3″-ethyl-5″-methylhex-3″-en-2″-yloxy)-2′-methylpropyl propionate and its cyclobutanecarboxylate leads to a synergistic increase in volume, muskiness and overall lushness. These two musks ideally complement each other in a way that otherwise can be observed only when a macrocyclic musk is combined with a linear alicyclic musk in so-called white-musk accords. As both odorants combine elements of macrocyclic and linear alicyclic musks, their synergistic effect is even more complex and natural, sweet, fruity, floral, seed-like, green and sappy, in addition to a dominant and sensual muskiness.

### EXAMPLE 11

### Functional perfume composition for use in shower gel featuring (3″E)-2′-(3″-ethyl-5″-methylhex-3″-en-2″-yloxy)-2′-methylpropyl propionate

| Compound/Ingredient | parts by weight 1/1000 |
|---|---|
| Calypsone (6-methoxy-2,6-dimethyloctanal) | 80 |
| (R)-(−)-Carvone | 1 |
| Citrathal R (main component: 1,1-diethoxy-3,7-dimethyl-2,6-octadiene) | 2 |
| Decanal | 1 |
| Dipropylene glycol (DPG) | 760 |
| Freskomenthe (2-(1′-methylpropyl)cyclohexanone) | 5 |
| Galbanone (1-(5′,5′-dimethyl-1-cyclo- | 6 |
| hexen-1-yl)-4-penten-1-one) at 10% in DPG | |
| Hedione HC (cis-methyl 2-(3′-oxo-2′-pentylcyclopentyl)acetate) | 25 |
| Herbanate (ethyl 3-isopropylbicyclo[2.2.1]hept-5-ene-2-carboxylate) | 20 |
| (2E,6Z)-Nona-2,6-dienal | 40 |
| Pharaone (2-cyclohexyl-1,6-heptadien-3-one) at 10% in DPG | 20 |
| (3″E)-2′-(3″-ethyl-5″-methylhex-3″- | 40 |
| en-2″-yloxy)-2′-methylpropyl propionate | |
| | 1000 |

At only 4%, the powerful, powdery musk (3″E)-2′-(3″-ethyl-5″-methylhex-3″-en-2″-yloxy)-2′-methylpropyl propionate provides the cosmetic musky foundation of this natural, green-watery scent when applied at 0.1% in shower gel. (3″E)-2′-(3″-Ethyl-5″-methylhex-3″-en-2″-yloxy)-2′-methylpropyl propionate combines very well with the cucumber character, rounds off and tones down the rather harsh green accord, and conveys caressing comfort and a musky feeling closely reminiscent of that of polycyclic musks.

### EXAMPLE 12

### Measurement of the vapour pressure and odour threshold of compounds of the invention in comparison with 2-methyl-2-[(1,2,4-trimethyl-2-pentenyl)oxy]propyl ester.

For each compound the vapour pressure was determined via the following procedure: 500 mg of the test compound was added to a headspace container which was then scaled. The container was incubated at constant 25° C. until the compound reached equilibrium between the gas and the liquid phase. An exactly defined but variable volume between 0.5-1.0 L of this saturated headspace was trapped on a micro filter using Porapak Q as sorbent. After filter extraction with 30-100 μl methyl tert.-butyl ether), an aliquot of the extract was analyzed by gas chromatograph (GC). Quantification was performed by the external standard calibration method. The concentration in the original headspace was calculated (in terms of μg/l) from the headspace volume sucked through the micro filter and the aliquot of the filter extract injected into the gas chromatograph. The final headspace concentration value for a given test compound was obtained as the mean value of three independent measurements.

Further information of the technique described hereinabove may be found in the article of F. Etzweiler, E. Senn, N. Neuner-Jehle, Ber. Bunsen-Ges. Phys. Chem. 1984, 88, 578.

For each compound the odour threshold value was determined by gas chromatograph (GC) detection. Different dilutions of the test compound were injected into a GC in descending order of concentration until a panelist failed to detect the respective substance at the sniffing port. Each panelist (panel of 5 persons) smelled blind and pressed a button upon perceiving an odor. If the recorded time matched the retention time, the sample was further diluted. The last quantity detected at the correct retention time was the panelist's individual odor threshold. The final threshold value for a given test compound was obtained as the mean value of the individual threshold levels.

Further information of the technique described hereinabove may be found in N. Neuner-Jehle, F. Etzweiler. ‘Measuring of Odors’, in P. Müller. D. Lamparsky (Eds,). ‘Perfumes. Art, Science and Technology’, Elsevier Appl. Science Publ., London. 1991: pp. 153.

Results are detailed in Table 1 below.

TABLE 1

| | Vapour | Odour |
|---|---|---|
| | Pressure | Threshold |
| Compound Name | μg/L | ng/L |
| (3″E)-2-(3,5-Dimethylhex-3-en-2-yloxy)-2- | 27.57 | 5.9 |
| methylpropyl cyclopropanecarboxylate | | |
| (3″E)-2′-(3″-Ethyl-5″-methylhex-3″-en-2″yloxy)-2′- | 10.61 | 2.2 |
| methylpropyl cyclopropanecarboxylate | | |
| (3″E)-2′-(3″-Ethyl-5″-methylhex-3″-en-2″-yloxy)-2′- | 4.76 | 0.63 |
| methylpropyl cyclobutanecarboxylate | | |

As can be seen from the figures displayed in Table 1, the compounds according to the invention have both lower vapour pressures and lower odour thresholds in comparison with the prior art compound (3″E)-2-(3,5-dimethylhex-3-en-2-yloxy)-2-methylpropyl cyclopropanecarboxylate.

1. A compound of formula (I) 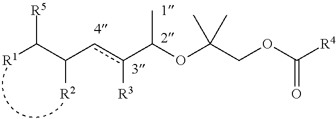
wherein R¹ and R² are independently selected from hydrogen, methyl, ethyl, propyl and isopropyl;
or R¹ and R² together form a saturated or monounsaturated 5- or 6-membered hydrocarbon ring, as represented by the arcuate dotted line;
R³ is selected from methyl and ethyl;
R⁴ is selected from methyl, ethyl, propyl, isopropyl, cyclopropyl, butyl, isobutyl, cyclobutyl and cyclopentyl;
R⁵ is selected from hydrogen and methyl, and the dotted line between positions 3″ and 4″ represents an optional double bond.

2. A compound according to claim 1, in which
R¹ is selected from hydrogen and methyl,
R² is selected from hydrogen and methyl,
or R¹ and R² together form a cyclohexenyl or cyclopentenyl ring;
R³ is selected from methyl and ethyl,
R⁴ is selected from ethyl, isopropyl, cyclopropyl and cyclobutyl.
R³ is selected from hydrogen and methyl, and
there is an (E)-configured double bond between C-3″ and C-4″.

3. A compound according to claim 1 selected from the group consisting of:
2′-Methyl-2′-(3′″-methylhex-3″-en-2″-yloxy)propyl cyclopropanecarboxylate,
2′-(3″-ethyl-5″-methylhex-3″-en-2″-yloxy)-2′-methylpropyl cyclopropanecarboxylate,
2′-(3″-ethyl-5″-methylhex-3″-en-2″-yloxy)-2′-methylpropyl propionate,
2′-(3″-ethyl-5″-methylhex-3″-en-2″-yloxy)-2′-methylpropyl cyclobutanecarboxylate.
2′-(3″,6″-dimethylhept-3″-en-2″-yloxy)-2′-methylpropyl cyclopropanecarboxylate,
2′-(3″,5″-dimethylhept-3″-en-2″-yloxy)-2′-methylpropyl cyclopropanecarboxylate,
and 2′-(4″-(cyclohex-3′″-enyl)-3″-methylbut-3″-en-2″-yloxy)-2′-methylpropyl isobutyrate.

4. (canceled)

5. A fragrance composition comprising one or more fragrance ingredients and at least one compound as defined in claim 1.

6. A method of creating, enhancing or modifying a fragrance composition by using alone as the fragrance composition, or in combination with at least one other fragrance ingredient in the fragrance composition, a compound according to claim 1.

7. A method for improving, enhancing, or modifying a fragrance application, comprising the addition therein of an effective amount of a compound as defined in claim 1.

8. A method according to claim 7 wherein the fragrance application is selected from the group consisting of perfumes, household products, laundry products, body care products and cosmetics.

9. A fragrance composition comprising one or more fragrance ingredients and at least one compound as defined in claim 3.

10. A method of creating, enhancing or modifying a fragrance composition by using alone as the fragrance composition, or in combination with at least one other fragrance ingredient in the fragrance composition, a compound according to claim 3.

11. A method for improving, enhancing, or modifying a fragrance application, comprising the addition therein of an effective amount of a compound as defined in claim 3.

12. A method according to claim 11 wherein the fragrance application is selected from the group consisting of perfumes, household products, laundry products, body care products and cosmetics.
